(19) United States
(12) Patent Application Publication
(10) Pub. No.: US 2023/0210488 A1
(43) Pub. Date: Jul. 6, 2023

(54) TISSUE MAPPING APPARATUS WITH EXTENDED RANGE AND METHOD THEREOF

(71) Applicant: Andrei Vertikov, Westwood, MA (US)

(72) Inventor: Andrei Vertikov, Westwood, MA (US)

(21) Appl. No.: 18/091,551

(22) Filed: Dec. 30, 2022

Related U.S. Application Data
(63) Continuation-in-part of application No. 17/990,673, filed on Nov. 19, 2022.
(60) Provisional application No. 63/295,623, filed on Dec. 31, 2021.

(51) Int. Cl. A61B 6/00; A61B 17/34; A61B 1/00; G06T 7/00

(52) U.S. Cl. CPC A61B 6/5217; A61B 17/3478; A61B 1/00096; G06T 7/0014

(57) ABSTRACT

A tissue mapping system includes: an imaging stylet, a stylet-deploying mechanism, and a system console with data-processing capability. This tissue mapping system calculates, in-real time, a position of the stylet during tissue imaging and sensing. This position is then used to combine and re-map image and sensor data acquired by the stylet from different regions of the mapped tissue. The stylet is configured to acquire image data within its vicinity when inserted in a tissue, and also has a sensing region along a flexible distal portion of its length. The stylet-deploying mechanism inserts the stylet in different regions of the mapped tissue iteratively. The stylet-deploying mechanism also incorporates features for registering the position of the stylet by using strain sensing or image data. The system console communicates with the stylet to calculate the position of the stylet by using intra-operative tissue image data and distributed strain data within the sensing region of the stylet. The stylet incorporates optical guides that are advantageously used both for imaging and for distributed strain sensing. Another aspect of the invention is the use of the very same imaging and strain sensing optical guide to interrogate biochemical sensors disposed distally within the stylet in some embodiments.

### CROSS-REFERENCE TO RELATED APPLICATIONS

This application is a Continuation-In-Part of U.S. patent application Ser. No. 17/990,673 filed on Nov. 19, 2022 entitled “Method and System for Image-guided Procedures with Sensing Stylet”, which claims the benefit of U.S. Provisional Application Ser. No. 63/281,913 filed on Nov. 22, 2021 entitled “Method and System for Image-guided Procedures with Sensing Stylet”, each of which is incorporated by reference herein in its entirety for all purposes.

This application also claims the benefit of U.S. Provisional Patent Application Ser. No. 63/295,623 filed on Dec. 31, 2021 entitled “Tissue Mapping Apparatus with Extended Range and Method Thereof”, which is incorporated by reference herein in its entirety for all purposes.

### TECHNICAL FIELD

The present invention relates to the field of diagnostic medical imaging and in-vivo tissue characterization and, more specifically, to minimally-invasive interstitial mapping of live tissue to diagnose a disease or to guide a therapy.

### BACKGROUND

Non-invasive and minimally-invasive imaging. The need for in-vivo imaging for medical diagnostics and for guidance and control of diagnostic, therapeutic and surgical procedures is well recognized. In-vivo imaging is also important in studies of live animals, for example for drug discovery or in fundamental research of disease biology. Existing non-invasive imaging modalities such as CT, ultrasound, or MRI often lack spatial resolution to map regions of interest within tissues of humans or animals with sufficient detail. On the other hand, current higher-resolution minimally-invasive imaging techniques such as, for example, Optical Coherence Tomography (OCT), ultrasound, confocal microscopy and alike cannot map sufficiently large regions of tissues. For example, the penetration depth of OCT is typically less than two millimeters when characterizing a cancerous tissue; such shallow imaging depth should be contrasted with tumor sizes of up to few centimeters that need to be evaluated in some clinical scenarios. Such a trade-off between the spatial resolution and the characterization depth limits the efficacy for in-vivo imaging making it important to increase its imaging range for high-resolution modalities.

Multi-modality. Often several imaging modalities need to be advantageously combined for effective in-vivo tissue characterization. For example, typing tissue within a tumor is challenging when relying only on structural information. More generally, knowledge of spatial distributions of various biochemical constituents and molecular characteristics within a tumor is important for effective selection and modulation of cancer treatment. Accordingly, there are enhanced imaging modalities such as, for example, fluorescence molecular tomography that add molecular and cellular maps to live tissue characterization. There are also techniques to characterize tissue using interstitially-placed biochemical sensors. Significant challenges exist, however, for integrating enhanced imaging and sensing modalities in a practical minimally-invasive device that can comprehensively map tissue properties with sufficient spatial resolution or range.

The present invention is intended to address these and several other deficiencies of minimally invasive tissue characterization as described below.

### SUMMARY OF OBJECTIVES AND EXEMPLARY EMBODIMENTS

Embodiments of the invention provide a tissue mapping system that includes: an imaging stylet, a stylet-deploying mechanism, and a system console with data-processing capability. Optionally, some embodiments also include a visualization sub-system to guide stylet deployment. This tissue mapping system calculates, in-real time, a position of the stylet during tissue imaging and sensing. This position is used to combine and re-map image and sensor data acquired by the stylet from different regions of the mapped tissue.

Main objective of the present invention is to provide high-resolution volumetric tissue mapping without compromising its mapping range or its invasiveness. Accordingly, in specific embodiments, a miniaturized imaging probe in the form of a flexible stylet is provided. The stylet is configured to acquire image data within its vicinity when inserted in a tissue. A stylet-deploying mechanism is also provided to insert the stylet in different regions of the mapped tissue iteratively. In addition, the stylet has a sensing region along a flexible distal portion of its length. A system console is also provided that communicates with the stylet to calculate the position of the stylet by using intra-operative tissue image data and distributed strain data within the sensing region of the stylet. The stylet incorporates, as a main aspect of the invention, optical guides that are advantageously used both for imaging and for distributed strain sensing, enabling miniaturization of the stylet for accomplishing the main objective of the invention. Another aspect of the invention is the use of the very same imaging and strain sensing optical guide to interrogate biochemical sensors disposed distally within the stylet in some embodiments. A stylet-deploying mechanism incorporating features for registering the position of the stylet by using strain sensing or image data is yet another aspect of the invention.

In one embodiment, the imaging stylet incorporates an eccentric rotatable guide of optical energy that couples proximal and distal ends of the stylet; the said rotatable optical guide is disposed within the stylet body with a lateral offset relative to the rotational axis of the guide and also relative to the neutral bending axis of the stylet. In operation, the optical guide rotates freely within the stylet to generate distal scanned patterns for tissue imaging using optical elements attached to the optical guide distally. At the same time, the system console measures, within a distal sensing region of the same rotating optical guide, a spatial distribution of time-varying strain modulated by the said rotation. The stylet position is then calculated by analyzing intra-operatively acquired image data and intra-operatively measured strain distribution data. In some related embodiments, the distal end of the stylet also has a transparent portion incorporating immobilized fluorophores that form a distal biochemical sensor interrogated optically via the rotatable optical guide. With redeployment of the stylet, the system console combines acquired image and sensor data from different tissue regions, remapping the data using the stylet position information to render 3D scenes with extended mapping range.

Other embodiments provide the imaging stylet that incorporates eccentric optical guides fixedly attached to the stylet body with lateral offsets relative to the neutral bending axis of the stylet. In these embodiments, each individual eccentric optical guide directs, using distal optical elements at the tip of the stylet, a portion of optical energy towards an imaged tissue thus forming an optical beam with a fixed spatial relationship with the stylet distal end. In related embodiments, the fixed optical guides are also disposed with axial offsets between some of the distal optical elements. In yet some other embodiments, the fixed optical guides communicate optically with portions of the distal end of the stylet that incorporate immobilized fluorophores; the said fluorophores form biochemical sensors interrogated optically via the fixed optical guides. At least in some embodiments, one common distal optical element directs optical energy towards an imaged tissue from a plurality of the fixed eccentrically-positioned optical guides. In some specific embodiments, the said common directing element is a curved mirror or a faceted mirror. Yet in other specific embodiments, the single common directing optical element is a wide-angle refractive lens or a diffractive metalens. In operation, the system console acquires one-dimensional (1D) image data sets from the individual fixed optical beams outcoupled from the corresponding optical guides and also measures strain distributions within sensing regions of at least some eccentrically positioned optical guides. The stylet position is then calculated by analyzing intra-operatively acquired 1D image data sets and intra-operatively measured strain distribution data. During repositioning of the stylet, the system console combines the acquired 1D image data sets, remapping the said 1D image data using the stylet position information to render 2D or 3D scenes of imaged tissue. With redeployment of the stylet, the system console combines acquired image and sensor data sets from different tissue regions, remapping the data using the stylet position information to render 3D scenes with extended mapping range.

Some other embodiments provide structures within the imaging stylet that integrate eccentrically positioned optical guides with a distal scanning mechanism to generate scanned patterns of optical energy emitted by the stylet towards an image tissue. In some specific embodiments, a piezo element is disposed distally between the optical waveguides and a distal optics of the the stylet to actuate X-Y scanning of a distal tip of an optical guide. In related embodiments, an optical energy guide with a stepped outer diameter structure of its distal end is provided to facilitate integration of the optical guide and a distal scanning arrangement in a miniaturized stylet. Additionally, at least in some related embodiments, a concentrating optical element is disposed between eccentric optical energy guides and a distal optics of the stylet to improve the collection efficiency for optical energy returned by an imaged tissue. Yet in some other specific embodiments, a torsional scanning arrangement is provided disposed distally within the stylet body. The said torsional scanning arrangement rotationally reciprocate distal ends of eccentrically-positioned optical guides to generate oscillating scanned patterns of optical energy outcoupled by the stylet for tissue imaging. In some related embodiments, a tubular structure with deposited coiled electrodes in operable communication with the console and a portion of an optical guide coated with a magnetic material form an electro-magnetically actuated torsional scanning arrangement. Additionally, at least in some related embodiments, the common directing optical elements mentioned above form beams of optical energy outcoupled towards a tissue from a plurality of eccentric optical guides scanned by a distal torsional scanning arrangement. A position of the stylet is then calculated by analyzing intra-operatively acquired image data and intra-operatively measured strain distribution data. In some embodiments, the system console remaps and re-renders intra-operatively acquired image data in accordance with the calculated stylet positions. With redeployment of the stylet, the system console combines acquired image and sensor data sets from different tissue regions, remapping the data using the stylet position information to render 3D scenes with extended mapping range.

In some embodiments, the stylet-deploying mechanism includes a template with guiding channels for iterative insertions of the stylet in different regions of the mapped tissue. Each guiding channels is also configured to induce known and pre-determined strain distributions in the stylet during the insertions. The said strain distributions are used to register positions of the stylet relative to the stylet-deploying mechanism. In some further embodiments, the guiding channels incorporate micro-bending features to induce known strain distributions in the stylet. In some other embodiments, the stylet-deploying mechanism includes an articulating arm that slideably accommodates the stylet for iterative insertions of the stylet in different regions of the mapped tissue. In some embodiments the said articulating arm is a motorized robot with associated control and user interface units. In some specific embodiments the articulating arm is formed by concentric continuously rotatable pre-bended tubes. The concentric tubes are also configured to induce known strain distribution in the stylet at least in some embodiments. In yet some other embodiments, the stylet-deploying mechanism includes a flexible needle and a guiding cannula slideably accommodating the stylet. The guiding cannula directs iterative insertions of the stylet in different regions of the mapped tissue by changing its bending angle and its rotational orientation relative to the flexible needle. In some embodiments, the bending angle is changed by sliding a pre-bended cannula within the needle. The guiding cannula and the flexible needle also have mating features to actuate distal rotation of the cannula when the cannula slides within the needle. Yet in some other embodiments, the guiding cannula integrates bending elements to change its bending angle for iterative insertion of the stylet in different regions of the mapped tissue. The guiding cannula is also configured to induce known and pre-determined strain distributions in the stylet at least in some embodiments.

Embodiments are also provided that combine all or some the features of the tissue mapping apparatus described above.

Methods of using the tissue mapping system of the present inventions to address the above objectives are also provided.

### BRIEF DESCRIPTION

The invention will be more fully understood by referring to the following Detailed Description in conjunction with the Drawings, of which:

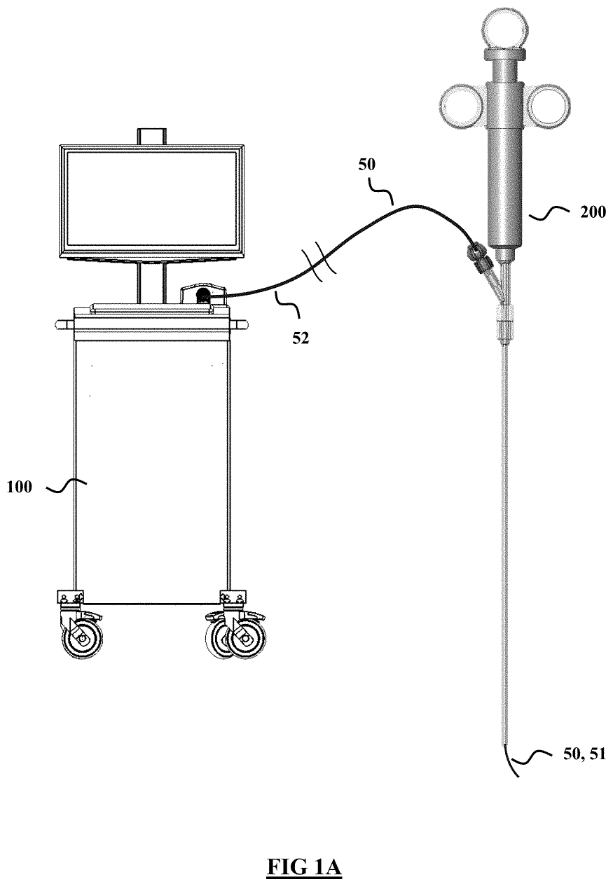

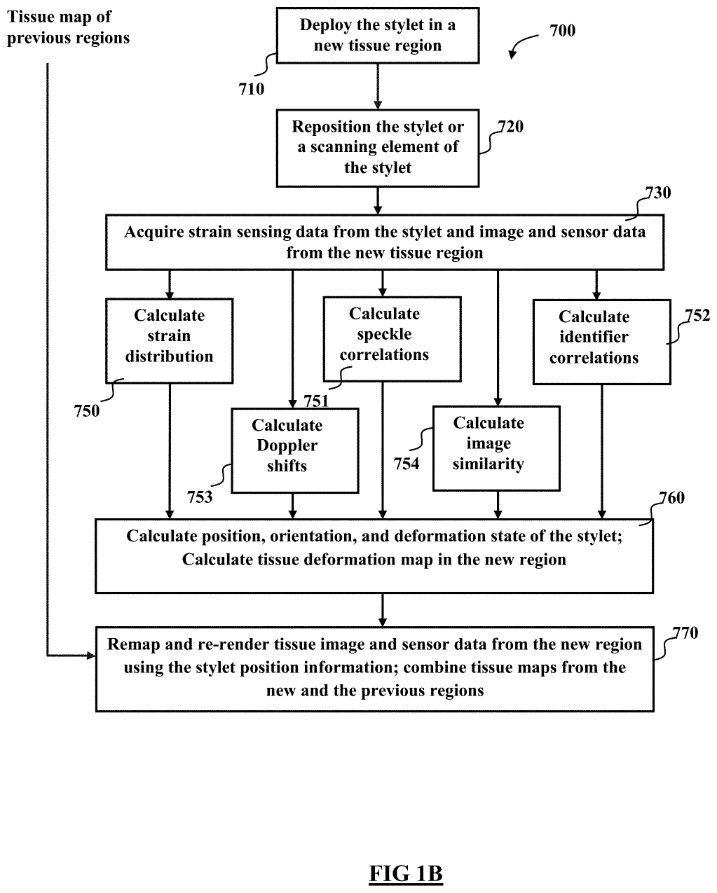

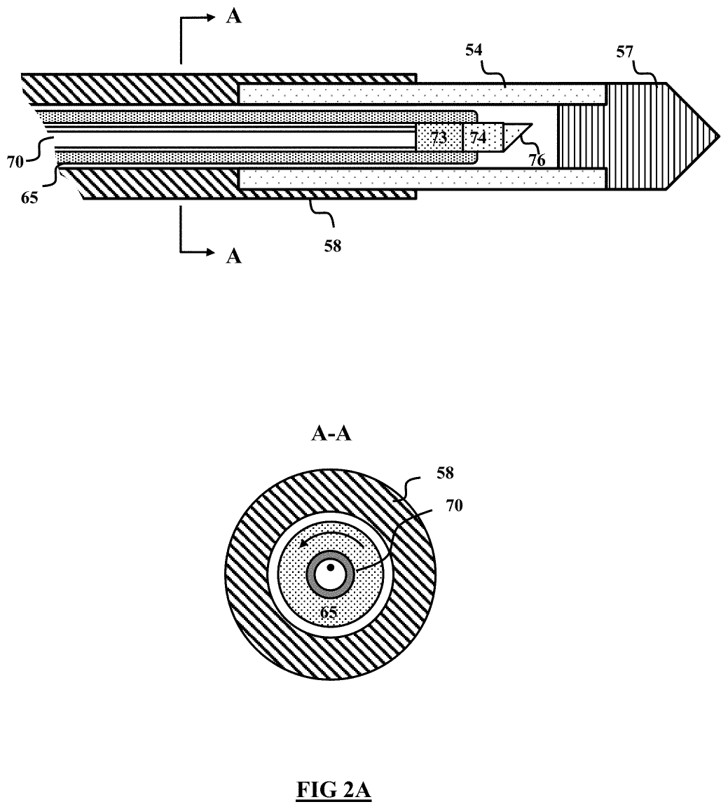

FIG. 1A is a top view of a tissue mapping system of the invention;

FIG. 1B is a flow chart of extending tissue mapping range using iterative deployment of the stylet in different tissue regions together with calculation of the stylet position based on the fusion of distributed strain sensing and image data acquired by the stylet in accordance with the method of the invention;

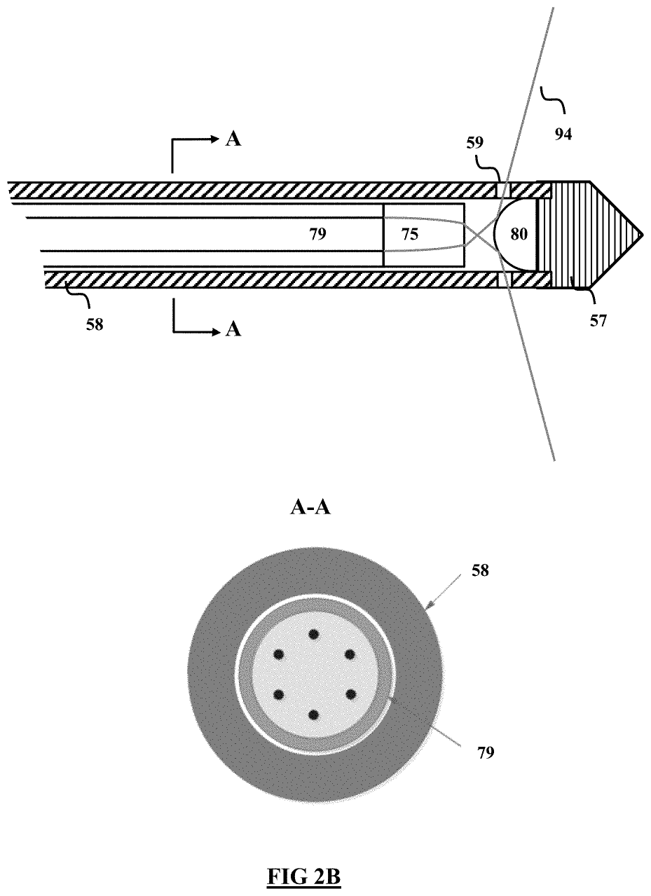

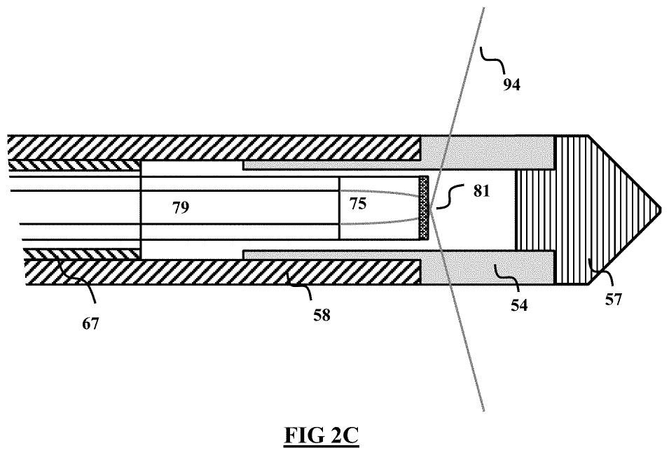

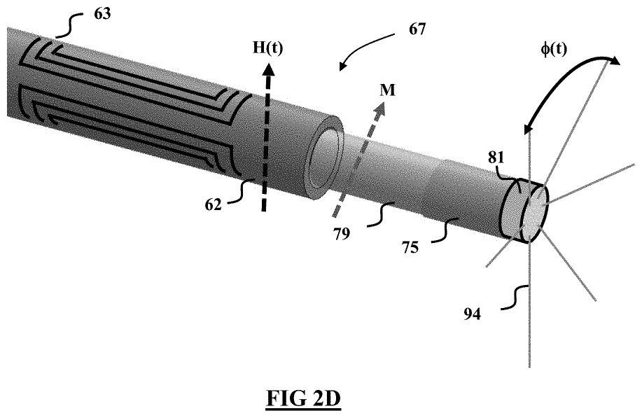

FIG. 2A shows partial sections of the imaging stylet with a rotatable strain-sensing optical channel in accordance with one example of the first preferred embodiment of the invention;

FIG. 2B shows partial sectional views of exemplary strain-sensing and imaging arrangements in the distal end of the imaging stylet in accordance with the second preferred embodiment of the invention;

FIG. 2C is a partial sectional view of exemplary strain-sensing and imaging arrangements in the distal end of the imaging stylet in accordance with the third preferred embodiment of the invention, wherein FIG. 2D shows an exemplary torsional scanning arrangement in accordance with this embodiment;

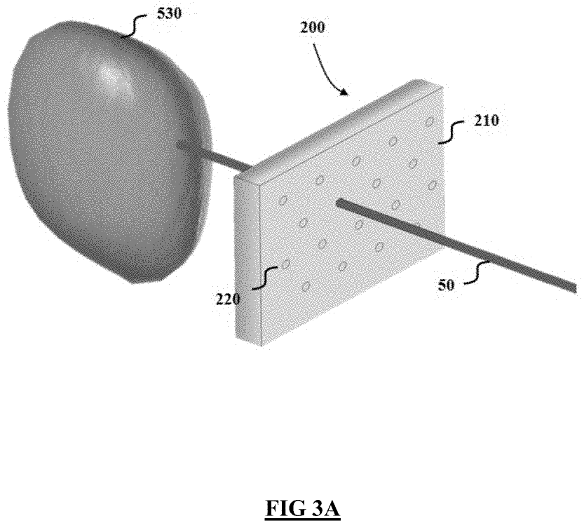

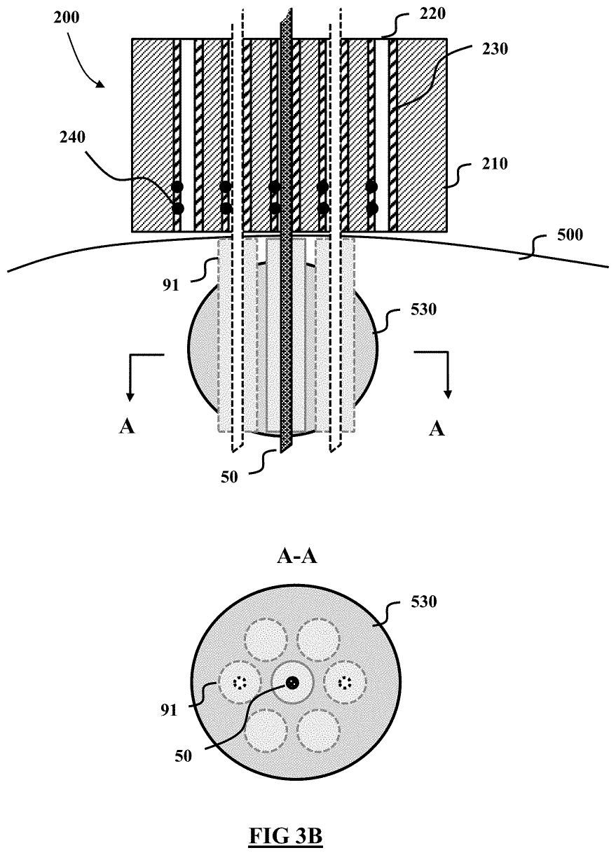

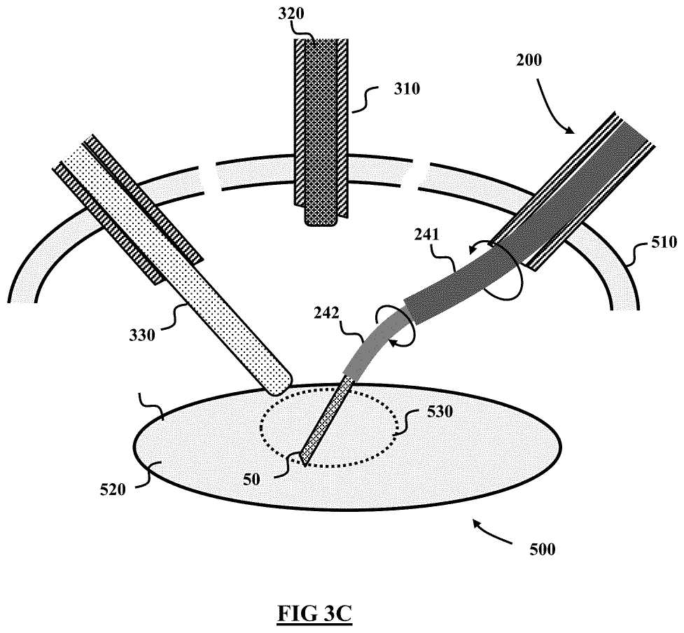

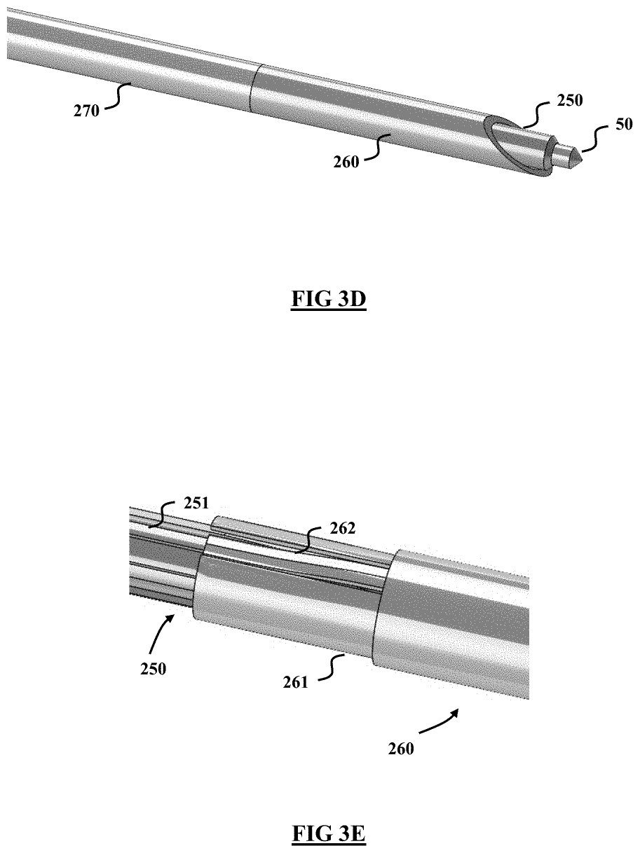

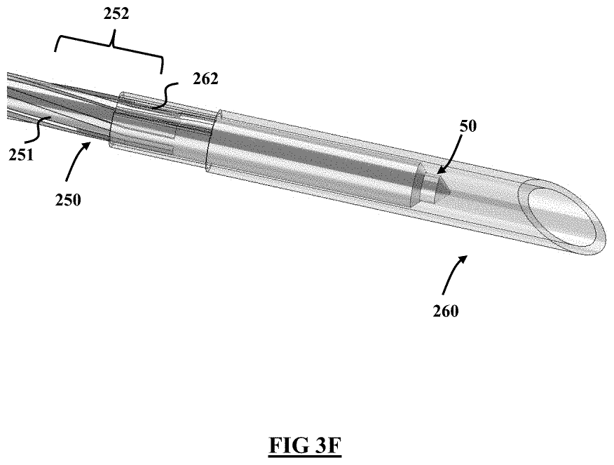

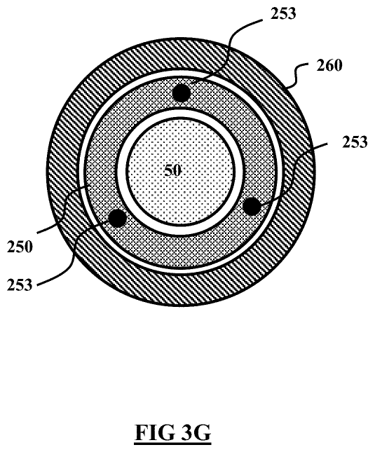

FIG. 3A shows the first exemplary embodiment of the stylet-deploying mechanism of the invention incorporating a stylet-guiding template, wherein FIG. 3B further shows partial sectional views of this embodiment;

FIG. 3C shows the second exemplary embodiment of the stylet-deploying mechanism of the invention incorporating an articulating arm;

FIG. 3D shows the third exemplary embodiment of the stylet-deploying mechanism of the invention incorporating a laterally deploying needle arrangement, wherein FIGS. 3E and 3F show detail views of this embodiment, whereas FIG. 3G shows an alternative laterally deploying needle arrangement incorporating distal bending elements;

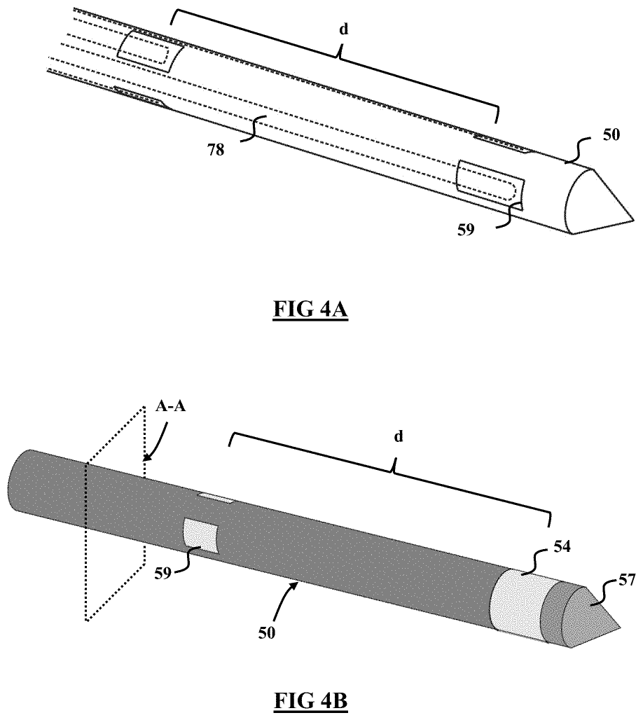

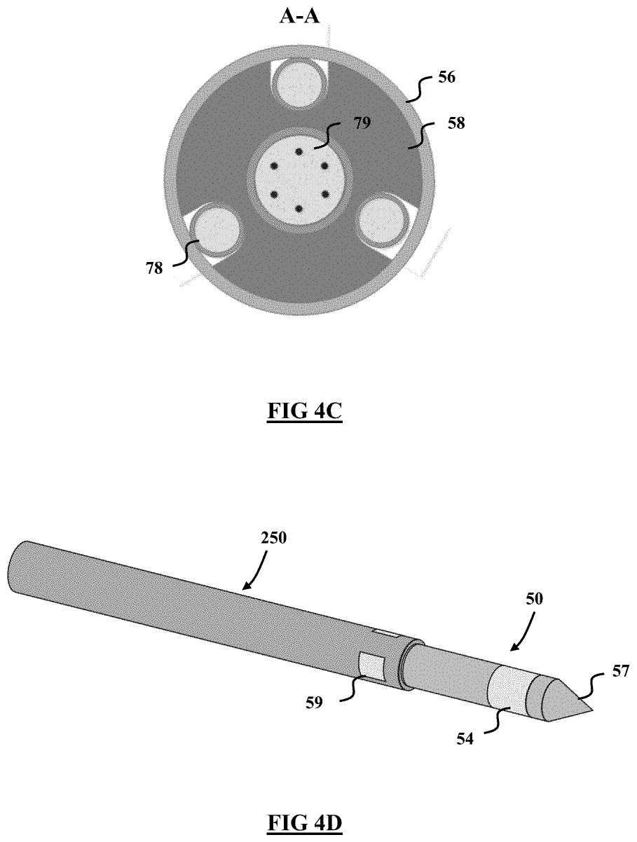

FIGS. 4A-4D show several exemplary embodiments of the stylet of the invention with enhanced biocomposition mapping capability;

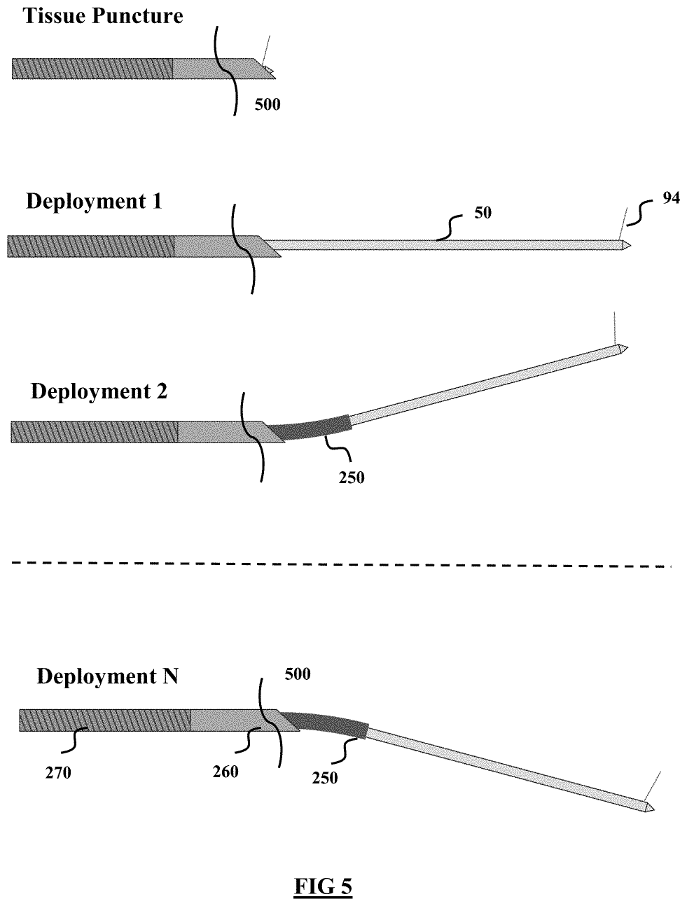

FIG. 5 illustrates steps of mapping a tissue using the method of the invention with an exemplary stylet-deploying mechanism of the invention.

### DETAILED DESCRIPTION

For clarity of the presentation, the following disclosure is structured subdivided as follows. The description associated with FIGS. 1A and 1B is the general description of the tissue mapping apparatus and the methods of the present invention. FIGS. 2A-2D and 3A-3G relate to several preferred embodiments of the imaging stylet and the stylet-deploying mechanism, respectively. FIGS. 4A-4D relate to several exemplary embodiments of the invention with enhanced biocomposition mapping. Finally, FIG. 5 describes an exemplary tissue mapping procedure with an associated use of an exemplary stylet-deploying mechanism in accordance with the method of the present invention.

### General Description.

FIG. 1A shows an exemplary embodiment of the tissue mapping system of the invention. The system has a flexible imaging probe 50 also referred to as an imaging stylet in this disclosure. The stylet 50 is configured to acquire image data intra-operatively. It is also configured to sense strain distribution in at least one portion of its elongated body. The system also contains a stylet-deploying mechanism or deployment instrument 200. The distal end of the imaging stylet is configured to be insertable in a lumen of the deployment instrument 200. During operation, the imaging stylet 50 is in communication at its corresponding proximal ends with a system console 100 that processes intra-operative image data and strain sensing data received from the stylet 50. Using the intra-operative data, the system console 100 calculates a position of the the stylet 50 in a tissue relative to the deployment instrument 200. Aggregately, the stylet 50, the deployment instrument 200, and the system console 100 are referred to as a tissue mapping system or a tissue mapping apparatus of the present invention. Optionally, in some embodiments, the tissue mapping system also includes auxiliary sub-system for visualization and guidance of stylet placement in a tissue as will be described in further detail below.

The term “distal ends” implies, in the context of the present disclosure, distal end portions of instruments intended to be placed inside or in close proximity to the mapped tissue. The term “proximal ends” implies, in the context of the present disclosure, the corresponding “opposite” portions of the instruments that are intended to be held and manipulated by an operator or to be interfaced with the system console 100. The term endoscope implies, in the context of the present disclosure, any flexible or rigid endoscopic imaging device or system such as a bronchoscope, a laparoscope, a surgical robotic system, an endoscopic robotic system and alike. Accordingly, endoscopic instruments imply, in the context of the present disclosure, both endoscopic instruments deployable via endoscopic working channels and surgical instruments deployable via separate surgical ports.

The terms “position”, “probe position”, “stylet position” imply, in the context of this disclosure, both a position and an angular orientation of the stylet distal end, unless the context clearly dictates otherwise. In addition, the term “or” is an inclusive “or” operator and is equivalent to the term “and/or”, unless the context clearly dictates otherwise. The term “real-time” implies, in the context of the present disclosure, substantially real-time, that is sufficiently fast so that an alignment of a probe or a stylet relative to an imaged scene is not lost due to uncontrolled tissue motion.

The term “intra-operative data” and related terms imply, in the context of this disclosure, image data and strain-sensing data acquired by the imaging stylet of the invention. The term “pre-operative data” and related terms imply either image data acquired by other imaging devices such as, for example, X-ray, CT, MRI, OCT, or ultrasound devices or image data acquired by the imaging probe of the invention in a previous imaging procedure or both, unless the context clearly dictates otherwise.

Referring again to FIG. 1A, the stylet 50 is an elongated flexible body characterized by its longitudinal axis, the stylet consisting of a distal end 51 and a proximal end 52 that are connected with a flexible shaft. When in operable communication with the device console, the stylet 50 projects an interrogating optical energy (understood to be UV, visible or NIR optical radiation with wavelengths in the range 0.2-2 μm) at its distal end 51 toward the ambient medium that may include an interrogated tissue. The stylet 50 also receives a returned optical energy originated at the tissue in response to the interrogating energy, the returned energy being encoded by the tissue response. Further details of the imaging stylet 50 and the system console 100 are described in U.S. patent application Ser. No. 17/990,673 incorporated by reference herein in its entirety. In addition to receiving the returned energy from the tissue region, the stylet 50 also receives a returned optical energy from a sensing region within its distal end. The returned optical energy from the sensing region of the stylet is processed by the console to obtain a strain distribution in the said region using the method of distributed optical sensing. This distributed strain data, together with tissue image data, is used by the console 100 to calculate a position of the stylet 50 in accordance with the method of the present invention as described in U.S. patent application Ser. No. 17/990,673. Additionally, at least in some embodiments, the stylet 50 incorporates biochemical sensors at its distal end as will be described in further details below.

Proceeding now to a detailed description of a tissue mapping process with the system of the invention, FIG. 1B shows an exemplary recursive sequence 700 that builds a combined tissue map using a tissue map of previous regions and image and sensor data acquired at a new tissue region upon a current deployment of the stylet. Specifically, the iterative process 700 includes a stylet deployment sub-process 710 when the stylet is inserted in a new region followed by repositioning of the stylet or its internal scanning elements in a sub-process 720. During the repositioning 720, image and sensor data from the new tissue region are acquired simultaneously with distributed strain sensing in an acquisition sub-process 730. The acquired strain sensing data is used to calculate strain distributions within the sensing region of the stylet in sub-process 750 as described in U.S. patent application Ser. No. 17/990,673. The acquired image data is used to calculate Doppler shifts (sub-process 753), speckle correlations (sub-process 751), image similarities (sub-process 754), and position identifier correlations (sub-process 752) as also described in U.S. patent application Ser. No. 17/990,673. A position of the stylet during repositioning 720 and data acquisition 730 is calculated in a sub-process 760 based on Extended or Unscented Kalman Filter or, more generally, on any Bayesian Filter described in detail in U.S. patent application Ser. No. 17/990,673 and references wherein. Optionally, the estimation sub-process 760 also calculates a deformation of the stylet and a deformation map of the tissue in the new region as will be described below. Finally, a sub-process 770 remaps and re-render combined image and sensor data using positional information calculated in the sub-process 760.

From the description below and in the references provided it is clear how the Kalman filter process can be modified to register positional information at each tissue region of the sequence 700 with a common position of the deployment instrument by including degrees of freedom of the said instrument in the position estimation sub-process 760. An exemplary modification of the sub-process 760 includes analyzing an instrument region in the image data using a process model incorporating a known spatial relationship between an imaged portion of the deployment instrument and the stylet. Alternatively or in addition, known and pre-determined strain distributions induced in the stylet by interacting with features and shapes within the stylet-deploying mechanism are included in the position estimation sub-process 760 to register a position of the stylet with the deployment instrument.

It is also clear from the references provided how an axial deformation of the stylet or an axial insertion force experienced by the stylet during deployments in a tissue is measured in the sub-process 760 at each position of the stylet. Alternatively or in addition, tissue deformation maps are independently estimated with the process of calculating the stylet position in a tissue as was described before in U.S. patent application Ser. No. 17/990,673. For example, Doppler shifts between sub-regions of image data are analyzed to generate tissue deformation flow, and then, by integrating, a tissue deformation map within the region. In case of speckle correlation analysis, translation vectors that maximize correlations of each block of image data correspond to tissue deformation vectors, once an average translation vector is subtracted. In another example, a non-rigid analysis of similarity of image data is used to determine deformation maps that are then included in a process model and a measurement model. Overall tissue motion relative to the stylet is independently estimated with image data acquired, while strain data is used to track position and orientation of the stylet relative to the deployment instrument. A prior known model of tissue deformations caused by an insertion of the stylet with a determined force is used to improve accuracy of tissue deformation mapping.

### Stylet Embodiments.

In reference to FIG. 2A, we now proceed to a detailed description of a first preferred embodiment of the system of the invention in which the stylet incorporates a sensing optical energy guide structured to be continuously rotatable inside a lumen of the stylet body. FIG. 2A shows two orthogonal cross-sectional views of the distal end of the stylet 50 where the rotatable optical energy guide of the stylet is an eccentric dual clad fiber (DCF) 70 co-axially attached to a torque coil 65 that rotates during imaging within an outer tube 58 that constitutes the stylet body. The outer body 58 is sealed distally with a transparent tube 54 and a rigid tip 57; the transparent tube 54 forms an imaging window for emitting and receiving optical energy. The eccentric DCF 70 is further fusion spliced, at its distal end, with a focusing element that aggregately consists of section of a coreless fiber 73 and a section of gradient index fiber 74. The distal focusing element has also an attached directing element 76 exemplified by a right angle prism in the specific embodiment of FIG. 2A. The inner, single mode, core of the DCF 70 is structured with a radial offset with respect to the DCF axis. Consequently, there is a radial offset between the inner core and a rotation axis of the DCF 70 and the torque coil 65 for the arrangement of FIG. 2A. Also, there is a radial offset between the inner core of the rotatable DCF 70 and a neutral bending axis of the stylet body. As a result of the eccentric offsets, the strain distribution S within the inner single mode core of the DCF in the strain sensing region becomes a time-varying function S(t,l) when the optical energy guide 70 rotates. Here t denotes time of strain measurement and l denotes position coordinate along the stylet length. Further details of the imaging stylet, the system console, and the stylet position estimation for this embodiment are given in U.S. patent application Ser. No. 17/990,673.

In some related embodiments, portions of the window 54 contain a chemically-sensitive material that undergoes changes in optical properties in response to a selective interaction with a targeted analyte (e.g. oxygen, pH and so on) in the mapped tissue. The optical properties include absorption, fluorescence, polarization, Raman scattering and alike that are interrogated via the DCF 70 and the distal optical elements 73, 74 and 76. An example of a chemical-sensitive material is a fluorophore immobilized in a polymer coated on the window 54; the immobilized reagent changing intensity, a spectrum, or a lifetime of its fluorescence emission due to an interaction with a targeted analyte. In some other related embodiments, the chemically-sensitive material incorporates a bio-recognition element such an antibody, antigen, protein, enzymes, nucleic acids or cells in general interacting with a target analyte and affecting a transducing fluorophore immobilized in portions of the window 54. Accordingly, the optical interrogation involves an excitation and a collection of fluorescence in the window 54 via the rotatable energy guide in these embodiments.

Proceeding now to describe a second preferred embodiment of the stylet of the invention, FIG. 2B shows two orthogonal cross-sectional views that include a multi-core fiber (MCF) 79, for example an MCF with six eccentric single-mode cores. The MCF 79 is fixedly enclosed in a flexible tube 58, for example in a nitinol tube. FIG. 2B also shows a single focusing element 75, for example a GRIN lens; the single focusing element is common for a plurality of optical cores. The said common focusing element is attached to the MCF 79 to focus the optical energy exiting from the eccentric cores of the DCF and to project the optical energy towards a curved mirror 80. The curved mirror 80 directs the optical energy towards a tissue through exit windows 59 in the tube 58 forming the plurality 94 of the side exiting beams. In this arrangement, a multi-faceted reflective prism can be used as a directing element instead of the curved mirror 80. In some related embodiments the windows 59 is sealed with a polymer incorporating a chemically-sensitive material for optical biochemical sensing as described above. In alternative arrangements for the second preferred embodiment, a wide-angle lens, for example a diffractive lens or a metalens, is attached to the focusing element 75 to direct optical energy to a tissue to form the side exiting beams 94 as described in more detail in U.S. patent application Ser. No. 17/990,673. U.S. patent application Ser. No. 17/990,673 also describes other alternative implementations of the second preferred embodiment based on separate DCFs eccentrically and fixedly disposed within the stylet body together with related arrangements in the system console and the stylet proximal end.

In a third preferred embodiment of the stylet, the system of the invention is structured to image tissue using distal scanning Referring first to a cross-sectional view of the distal end presented in FIG. 2C, a torsional actuator 67 is attached distally to the outer tube 58 of the stylet. The said torsional actuator holds the MCF 79 having the eccentric cores as described before. Optically coupled to the distal end of the MCF 79 are the previously described focusing element 75 and the wide-angle diffractive directing element 81 that form an arrangement outputting the plurality of the side-exiting beams 94. In the specific example of the FIG. 2C, the plurality 94 is directed towards a tissue via a transparent distal tube 54, for example via a polyimide tube attached distally to the outer tube 58 and sealed with the cap 57. Also, in this specific example the focusing element 75 and the directing element 81 are attached to the distal end of MCF 79. In alternative arrangements, the focusing element or the directing element can be attached to the outer body or the cap of the stylet thus decoupling the said elements mechanically from the MCF 79. Alternatively or in addition, a curved or faceted mirror can be used as a directing element in these arrangements. During imaging, the torsional actuator 67 reciprocally rotates the distal end of the MCF 79 and, correspondingly, the plurality of beams 94 around the axis of the stylet, preferably with an amplitude of the reciprocal rotation ϕ(t) exceeding the angular separation of the side-exiting beams as shown in FIG. 2D that illustrates the actuator 67 in further details. Referring again to FIG. 2D, an exemplary distal end structure with the torsional scanner 67 has a polyimide tube 62 holding the DCF 79 with the attached focusing element 75 and the directing element 61. The tube 62 is further attached to the outer body 58 (not shown in FIG. 2D for clarity) of the stylet while the distal end of the MCF 79 is free to rotate within the tube 58. Electroplated copper coils 63 are deposited distally on the outer surface of the polyimide tube and covered with isolating material, for example with an additional layer of polyimide tubing, the said copper coils are connected proximally with the system console. The distal tip of the DCF 79 is also coated with a magnetic material magnetized to have a permanent magnetization vector M oriented at an angle with respect to the axis of the MCF as shown in FIG. 2D. During imaging, the console supplies an alternating electrical current to the coils 63 by to generate a time-varying magnetic field H(t) at the distal end of the stylet. The said magnetic field interacts with the magnetization vector M of the MCF 79 causing reciprocal oscillation of the MCF tip around its axis as a result. Accordingly, the plurality of beams 64 oscillate around the stylet axis to construct image data while positions of the beams together with the overall position of the distal end of the stylet are tracked in accordance with the methods of the invention using the process 700 of FIG. 1B. Alternatively, in a simplified distal end arrangement of the third preferred embodiment of the stylet, standard electro-magnetic coils are positioned externally to the patient body to generate a time-varying magnetic field for reciprocal oscillations of the magnetized distal tip of the MCF, thus making it unnecessary to fabricate the electroplated coils 63 in the stylet. In some related embodiments, portions of the window 54 incorporate a chemically-sensitive material for the above-described optical biosensing of the mapped tissue. Further examples of distally scanning mechanisms of the stylet that are in scope of the third preferred embodiments are given in the references provided, including a description of related arrangements in the system console and the proximal end of the stylet.

### Examples of Deployment Instruments.

Proceeding now to a detailed description of a first exemplary embodiment of the stylet-deploying mechanism of the invention, the deployment instrument 200 incorporates a template 210 with a plurality of channels 220 that guide insertions of the stylet 50 towards a tissue target 530 as shown in FIG. 3A. Partial cross-sectional views of FIG. 3B provide further details of mapping the tissue target 530 in a tissue 500 in a specific example when the stylet-guiding template 210 is a plate with a plurality of parallel through holes exemplifying the guiding channels 220. Preferably, the exemplary embodiment of the deployment mechanism 200 illustrated in FIGS. 3A and 3B is used when the target 530 is accessible from the outside of the tissue 500. In other words, the target 530 is within a reach of the stylet when the template 210 is placed in external contact with the tissue 500. Such reachable placement is possible, for example, in transperineal comprehensive mapping of the whole prostate organ for localization and grading of cancerous lesions to guide their focal treatment. Another example of a target being accessible from the outside of the tissue is a subcutaneous oncology model in a live animal. In some related embodiments, an auxiliary visualization instrument, for example an ultrasound probe, is used to align and to register the template with respect to the target. Alternatively or in addition, the auxiliary visualization instrument guides insertions of the stylet in the mapped tissue. Referring again to FIG. 3B, when the stylet 50 is repeatedly inserted and pulled back in each guiding channel 220, a plurality of volumetric data sets from regions 91 is acquired as illustrated in the cross-section A-A of FIG. 3B. Using positional information obtained with the method of the invention, the data sets from regions 91 are remapped and re-rendered to form a combine data set that aggregately characterizes a portion of the target 530 significantly larger than each individual region 91. In some embodiments the stylet is inserted in the guiding channels manually, whereas in some other embodiments the deployment instrument 200 includes a motorized manipulator for repeatable insertion of the stylet in the guiding channels 220. Yet, in some other embodiments the whole template 210 or its portion containing at least one channel 220 are displaced with a motor for iterative insertion of the stylet in different regions of the mapped tissue.

Also, at least in some embodiments, the guiding channels accommodate removable guiding tubes or cannulas 230 to accept stylets or other instruments of different dimensions, for example to accept medical instruments for biopsy or for focal treatment of the mapped tissue. The said medical instruments include devices for RF ablation, electroporation ablation, pulse field ablation, and cryogenic ablation. The guiding cannulas incorporate micro-bending features 240 to induce pre-determined distributions of strain in the inserted stylet to register positions of the stylet with respect to the template 210. In other words, when the stylet is inserted in a cannula 230, a strain distribution within the stylet has a prior known pattern with known distances between at least some maxima, the said distances are determined by locations of the micro-bending features 240 within the cannula. Accordingly, position of the stylet relative to the template 210 is determined by identifying the said prior known strain pattern within the overall strain distribution. In some related embodiments, the micro-bending features are disposed directly within the guiding channels. In some embodiments, the micro-bending features 240 are dents in the guiding cannulas. In a specific embodiment shown in FIG. 3B, the micro-bending features 240 are pins inserted in slots of the guiding cannula 230 orthogonally to the guiding channels 220. The micro-bending pins are sized to engage the inserted stylet without completely blocking its insertion in this specific embodiment. In some other related embodiment, the channels 220 are structured to deviate from a straight cylindrical shape imposing a known pre-determined strain distribution within the stylet during its insertion.

Referring now to FIG. 3C, a second exemplary deployment mechanism is preferably used to map a tissue 500 located within an anatomical or a surgically created cavity accessible percutaneously with trocars or needles 310 via skin area 510. In this embodiment, the deployment mechanism 200 includes an articulating arm to steer the imaging stylet 50 for iterative insertions in different regions of a tissue target 530. In a specific example of FIG. 3B, the articulating arm consists of a pre-bended tube 242 coaxially inserted in another pre-bended tube 241. The coaxial arrangement, also called concentric in the art of continuous robots, directs deployments of the stylet 50 to different regions of the mapped tissue with proximal rotations of the tubes 241 and 242. At least in some embodiments the pre-bended tube 242 has the micro-bending features described above for registering positions of the stylet with respect to the deployment instrument. Alternatively or in addition, a known shape assumed by the coaxial arrangement of the tubes 242 and 241 at each deployment position is used to register positions of the stylet; the known shape is determined by orientations of the concentric tubes at their proximal ends. The specific embodiment of FIG. 3C also shows an endoscope 320 and an auxiliary instrument 330 to visualize deployments of the stylet or to perform further diagnostic or therapeutic procedures with the mapped tissue. One example of the auxiliary instrument is an endoscopic ultrasound probe.

Referring now to FIGS. 3D-3G, a third exemplary deployment instrument is preferably used to map a tissue accessible from a narrow lumen within a patient body. In this embodiment, with its distal end illustrated in FIG. 3D, the deployment instrument incudes a flexible needle consisting of a distal tip 260 and a shaft 270. Slideable within the flexible needle is a flexible guiding cannula 250 that accepts the stylet 50. For example, the guiding cannula 250 is a nitinol tube. The cannula 250 has a pre-bended shape to deploy the stylet at a lateral angle relative to an axis of the needle tip when the cannula 250 protrudes from the tip 260. FIG. 3E provides further details how the needle tip 260 and the cannula 250 interact in a sliding arrangement for a lateral, off-axis, stylet deployment. In a specific example shown in FIG. 3E, the tip of the needle has a reduced outer diameter region 261 for bonding the needle shaft (not shown in FIG. 3E) using adhesive or fusion process. Furthermore, the needle tip also has a keying feature 262 that engages a corresponding mating feature 251 in the cannula. For example, the keying feature 262 is a bended lip machined in the region 261 and the engaging mating feature 251 is six grooves distributed equally on the circumference of the cannula. As further shown in FIG. 3F, the said grooves 251 are twisted distally within a region 252 in this specific embodiment. Accordingly, when the cannula 250 is pushed forward within the tip 260, the lip 262 engages the grooves 251 to rotate the cannula distally by approximately 60 degree in this specific example. In contrast, when the cannula is pulled back, the lip does not engage the grooves so that the cannula slides without rotation. Accordingly, by sliding the cannula axially the stylet is deployed in different regions of the mapped tissue. At least in some embodiments, the cannula 250 has the micro-bending features for registering positions of the stylet with respect to the deployment instrument. Alternatively or in addition, a known and pre-determined shape assumed by the cannula at each deployment position is used to register positions of the stylet.

In some alternative embodiments, the guiding cannula 250 incorporates at least one distal bending element 253 disposed along a portion of the cannula length as shown cross-sectionally in FIG. 3G that exemplify a nested arrangement of the needle distal tip 260, the guiding cannula 250 and the stylet 50 in these embodiments. The said bending elements 253 change the distal shape of the cannula 250 thus changing a lateral off-axis deployment angle of the stylet. In some specific embodiments, the bending elements are pull wires actuated proximally to bend the distal end of the cannula for an off-the-axis deployment of the stylet. In some other specific embodiments, the bending elements are shape-memory alloy (SMA) elements heatable with a current delivered to the cannula by the system console. Accordingly, the SMA elements bend the distal end of the cannula 250 to deploy the stylet with a lateral angle. In some other specific embodiments, the SMA elements are heated with an optical energy delivered via guides with the cannula as described in U.S. patent application Ser. No. 16/300,475 incorporated herein in its entirety. In some related embodiments, the needle tip 260 itself has a pre-bended shape for a lateral deployment of the stylet or has incorporated distal bending elements.

Embodiments with Enhanced Biocomposition Mapping.

The stylet and the console described in this disclosure and in the references provided present an apparatus capable of biocomposition mapping by collecting and analyzing spectral information from a tissue using the methods of diffuse reflectance spectroscopy, fluorescence, Raman scattering including surface enhanced Raman scattering and alike. Here we describe several examples of improved biocomposition mapping with the apparatus of the invention. In the first example, FIG. 4A shows the stylet 50 incorporating DCF guides 78 outcoupling optical energy via windows 78 displaced with an axial separation d between emitting and receiving windows. Accordingly, a biocomposition mapping with a deeper penetration (approximately d/3) is possible with this arrangement using diffuse reflectance spectroscopy. Alternatively or in addition, at least some portions of the windows 59 incorporate a chemically-sensitive material interrogated optically with the guides 78 for the biochemical sensing described above.

FIGS. 4B and 4C show a 3D view and a cross-sectional view, respectively, of the stylet distal end in another exemplary embodiment with enhanced biocomposition mapping. In this embodiment, the previously described DCF guides 79 receive optical energy via windows 59; the windows 59 disposed with an axial offset d relative to a transparent tube 54 that transmits optical energy from an MCF guide 79. As a result of the axial offset, a biocomposition tissue mapping with a deeper penetration is possible when optical energy is emitted by the MCF 79 and collected with the DCFs 79 via the windows 59. In the specific example of FIGS. 4B and 4C, the MCF 79 and DCF 78 are disposed within an inner lumen and outer groves of a stylet body 59, respectively, covered with a protective layer 56. In some related embodiments, the arrangement of FIG. 4B includes the distal torsional scanning mechanism described above. Alternatively or in addition, at least some portions of the windows 59 or 54 incorporate a chemically-sensitive material interrogated optically with the guides 78 or 79 for the biochemical sensing described above.

In an exemplary embodiment of FIG. 4D, optical guides and associated optical windows 59 are disposed within the slideable cannula 250 of the stylet-deploying mechanism. In this arrangement, biochemical tissue mapping is improved by varying a distance between a window 54 that emits optical radiation from the stylet 50 and the windows 59 that receive optical radiation in the cannula 250 while acquiring image data using the methods of diffuse reflectance spectroscopy.

### Exemplary Tissue Mapping Procedure.

Proceeding to explain further the method of the invention, details of an exemplary tissue mapping procedure are provided. This procedure is presented in reference to FIG. 5 and in further reference to FIGS. 3D, 3E, and 3F. Referring first to FIG. 5, the previously described third exemplary deployment instrument based on a flexible needle brings the stylet to the vicinity of a target tissue; the flexible needle delivered and manipulated with an endoscope or an endoscopic robot (not shown). First, the tissue is punctured with the flexible needle while the needle tip 260 fully encloses the guiding cannula 250 and the stylet 50. In a first deployment step, the stylet 50 extends towards the target tissue. Then, the stylet is pulled back inside the needle tip while image and sensor data are acquired. In a second deployment step, the pre-bended cannula extends from the needle tip to assume a curved shape; a lateral bending angle of the cannula determined by its protrusion from the needle tip. Further in the second deployment step, the stylet extends from the cannula for an off-axis insertion with respect to an axis of the previous deployment. Then, the stylet is pulled back into the needle tip while acquiring image and sensor data from a new tissue region. For a next deployment step, the cannula is pulled back to engage the rotating features 262 and 251 shown in FIG. 3E and FIG. 3F. As a result, an angular orientation of the curved shape of the cannula changes when the cannula is extended. The deployment steps are iterated to insert the style in different regions of the target tissue. Finally, a combined image and sensor data set is generated by remapping and the data acquired from the different regions using positional information as described above. The combined tissue map is then used to guide a biopsy or a focal treatment of the target tissue or to inform a systemic therapy.

### Other Considerations

It is to be understood that no single drawing used in describing embodiments of the invention is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

References throughout this specification have been made to “one embodiment,” “an embodiment,” “a related embodiment,” or similar language. Such references mean that a particular feature, structure, or characteristic described in connection with the referred to “embodiment” is included in at least one embodiment of the present invention. Thus, appearances of the phrases “in one embodiment,” “in an embodiment”, and similar language throughout this specification may, but do not necessarily, all refer to the same implementation of the inventive concept. Furthermore, the phrases “in another embodiment” and “in some other embodiments” as used herein do not necessarily refer to a different embodiment, although it may. Thus as described below, various embodiments of the invention may be readily combined, without departing from the scope or spirit of the invention It is to be understood that no portion of disclosure, taken on its own and in possible connection with a figure, is intended to provide a complete description of all features of the invention.

The invention as recited in claims appended to this disclosure is intended to be assessed in light of the disclosure as a whole, including features disclosed in prior art to which reference is made.

While the invention is described through the above-described exemplary embodiments, it will be understood by those of ordinary skill in the art that modifications to, and variations of, the illustrated embodiments may be made without departing from the disclosed inventive concepts. Furthermore, disclosed aspects, or portions of these aspects, may be combined in ways not listed above. Accordingly, the invention should not be viewed as being limited to the disclosed embodiment(s).

### References

Ref 1: Andrei Vertikov, U.S. patent application Ser. No. 17/990,673.

Ref 2: Andrei Vertikov, U.S. patent application Ser. No. 16/300,475.

What is claimed is:

1. A tissue mapping system comprising:
A first arrangement configured to image a tissue, the first arrangement including:
an elongated flexible body having a proximal end and an opposite distal end, and having an internal lumen extending from the proximal end to the distal end,
an optical guide extended inside the flexible body and configured to deliver an optical energy between the proximal end and the distal end, and also configured to return a portion of the optical energy from a sensing portion of the optical guide,
wherein the optical guide is also configured to be continuously rotatable inside the internal lumen of the first arrangement around a rotation axis; and
at least one optical directing element disposed within the distal end of the first arrangement and configured to transmit the optical energy delivered by the optical guide to the tissue;
a second arrangement configured to to guide the first arrangement to different regions of a mapped tissue; and
a system console including a data-processing unit with memory; the system console being in operable communication with the optical guide and configured to:
process the optical energy acquired from the tissue by the first arrangement and delivered by the optical guide to generate image data,
process the optical energy returned by the sensing portion of the optical guide to measure strain distribution data within the sensing portion,
calculate a position of the distal end of the first arrangement relative to the second arrangement using the strain distribution data, and
combine the image data from different tissue regions and remap the said image data using the calculated position.

2. A tissue mapping system according to claim 1, wherein the position calculation further uses the image data acquired by the first arrangement.

3. A tissue mapping system according to claim 1, wherein the optical guide is disposed with a lateral offset with respect to the rotation axis.

4. A tissue mapping system according to claim 1, wherein the second arrangement includes a plurality of guiding channels.

5. A tissue mapping system according to claim 1, wherein the second arrangement is a steerable arm configured to accept the first arrangement.

6. A tissue mapping system according to claim 1, wherein the second arrangement comprises a flexible needle configured to deploy the first arrangement at a lateral angle with respect to a distal tip of the flexible needle.

7. A tissue mapping system comprising:
A first arrangement configured to image a tissue, the first arrangement including:
an elongated flexible body having a proximal end and an opposite distal end, and having a longitudinal axis extending from the proximal end to the distal end,
a plurality of optical guides extended inside the flexible body and configured to deliver optical energy between the proximal end and the distal end, at least some of the optical guides also configured to return portions of the optical energy from sensing portions of the said optical guides,
wherein the optical guides are immovable affixed to the flexible body and at least some of the optical guides are positioned with lateral offsets with respect to the longitudinal axis; and
at least one optical directing element disposed within the distal end of the first arrangement and configured to transmit the optical energy delivered by the optical guides to the tissue;
a second arrangement configured to accept and to guide the first arrangement to different regions of a mapped tissue; and
a system console including a data-processing unit with memory; the system console being in operable communication with the plurality of the optical guide and configured to:
process the optical energy acquired from the tissue by the first arrangement and delivered by the optical guides to generate image data,
process the optical energy returned by the sensing portions of the optical guides to measure strain distribution data within the sensing portions,
calculate a position of the distal end of the first arrangement relative to the second arrangement using the strain distribution data and
combine the image data from different tissue regions and remap the said image data using the calculated position.

8. A tissue mapping system according to claim 7 further comprising a common optical directing element disposed within the distal end of the first arrangement and configured to transmit the optical energy delivered by a plurality of the optical guides to the tissue.

9. A tissue mapping system according to claim 8 wherein the common optical directing element is a wide-angle lens arrangement.

10. A tissue mapping system according to claim 7, wherein the position calculation further uses the image data acquired by the first arrangement.

11. A tissue mapping system according to claim 7, wherein the second arrangement includes a plurality of guiding channels.

12. A tissue mapping system according to claim 7, wherein the second arrangement is a steerable arm configured to accept the first arrangement.

13. A tissue mapping system according to claim 7, wherein the second arrangement comprises a flexible needle configured to deploy the first arrangement at a lateral angle with respect to a distal tip of the flexible needle.

14. A tissue mapping system comprising:
A first arrangement configured to image a tissue, the first arrangement including:
an elongated flexible body having a proximal end and an opposite distal end, and and having a longitudinal axis extending from the proximal end to the distal end,
a plurality of optical guides extended inside the flexible body and configured to deliver optical energy between the proximal end and the distal end, at least some of the optical guides also configured to return portions of the optical energy from sensing portions of the said optical guides,
wherein at least some of the optical guides are positioned with lateral offsets with respect to the longitudinal axis;
a scanning mechanism disposed within the distal end with at least some of the optical guides of the plurality affixed to the scanning mechanism; the scanning mechanism configured to scan the affixed optical guides; and
at least one optical directing element disposed within the distal end of the first arrangement and configured to transmit the optical energy delivered by the optical guides to the tissue;
a second arrangement configured to guide the first arrangement to different regions of a mapped tissue; and
a system console including a data-processing unit with memory; the system console being in operable communication with the optical guide and the scanning mechanism and configured to:
process the optical energy acquired from the tissue by the first arrangement and delivered by the optical guide to generate image data,
process the optical energy returned by the sensing portion of the optical guide to measure strain distribution data within the sensing portion,
calculate a position of the distal end of the first arrangement relative to the second arrangement using the strain distribution data, and combine the image data from different tissue regions and remap the said image data using the calculated position.

15. A tissue mapping system according to claim 14, wherein the position calculation further uses the image data acquired by the first arrangement.

16. A tissue mapping system according to claim 14, wherein the scanning mechanisms is a lateral scanning arrangement configured to scan the affixed optical guides laterally with respect to the longitudinal axis.

17. A tissue mapping system according to claim 14, wherein the scanning mechanisms is a torsional scanning arrangement configured to rotationally reciprocate the affixed optical guides around the longitudinal axis.

18. A tissue mapping system according to claim 14 further comprising a common optical directing element disposed within the distal end of the first arrangement and configured to transmit the optical energy delivered by a plurality of the optical guides to the tissue.

19. A tissue mapping system according to claim 14, wherein the second arrangement includes a plurality of guiding channels.

20. A tissue mapping system according to claim 14, wherein the second arrangement is a steerable arm configured to accept the first arrangement.

21. A tissue mapping system according to claim 14, wherein the second arrangement comprises a flexible needle configured to deploy the first arrangement at a lateral angle with respect to a distal tip of the flexible needle.

22. A method of mapping a tissue using a system having an imaging stylet, a deployment instrument, and an imaging console; the stylet including an optical guide configured to deliver imaging optical energy between the imaging console and the tissue, the deployment tool configured to guide the imaging stylet, and the imaging console communicating with the imaging probe, the method comprising:
(i) guiding a distal end of the imaging stylet towards a target in a tissue with the deployment instrument;
(ii) advancing the distal end of the imaging stylet from the deployment instrument and inserting a distal end of the imaging stylet in the tissue;
(iii) repositioning the distal end of the imaging stylet in the tissue and acquiring image data of at least a portion of the tissue;
(iv) measuring a strain distribution data within a sensing portion of the optical guide by processing optical energy returned from the sensing portion;
(v) determining position and orientation of the distal end of the imaging stylet relative to the deployment instrument by processing the image data and the strain distribution data acquired by the imaging stylet:
(vi) guiding the distal end of the imaging stylet to different regions of the tissue and repeating steps (iii)-(v) in each region;
(vii) combining the image data from each region and remapping the image data using the positional information determined in step (v).

23. A method of mapping a tissue according to claim 22, wherein determining position and orientation of the distal end of the stylet further comprising a comparison of the strain distribution data with a reference strain distribution data derived from a known structure of the deployment instrument.

24. A method of mapping a tissue according to claim 22, wherein the deployment instrument further comprising a flexible needle; the distal end of the imaging stylet inserted at an angle with respect to a distal end the flexible needle at least in some of regions of the mapped tissue.

25. A method of mapping a tissue according to claim 22, further involving collecting sensing data with the optical guide of the imaging stylet from a chemically-sensitive material disposed in the distal end of the imaging stylet.
